(19) United States
(12) Patent Application Publication
(10) Pub. No.: US 2016/0250159 A1
(43) Pub. Date: Sep. 1, 2016

(54) COMPOSITION, USE THEREOF, AND METHODS OF MANUFACTURING

(71) Applicant: Christopher Fisher Goldblatt, Santa Barbara, CA (US)

(72) Inventor: Christopher Fisher Goldblatt, Santa Barbara, CA (US)

(21) Appl. No.: 15/048,613

(22) Filed: Feb. 19, 2016

Related U.S. Application Data
(63) Continuation of application No. 14/863,881, filed on Sep. 24, 2015.
(60) Provisional application No. 62/054,497, filed on Sep. 24, 2014.

(51) Int. Cl. A61K 31/122; A61Q 19/08; A61K 8/35; A61K 45/06

(52) U.S. Cl. CPC A61K 31/122; A61K 45/06; A61Q 19/08; A61K 8/355

(57) ABSTRACT

A composition that includes at least two of: echinochrome A (2-ethyl-3,5,6,7,8-pentahydroxy-1,4-naphthoquinone); spinochrome A (2-acetyl-3,5,6,8-tetrahydroxy-1,4-naphthalenedione); spinochrome B (2,3,5,7-tetrahydroxy-1,4-naphthalenedione); spinochrome C (2-acetyl-3,5,6,7,8-pentahydroxy-1,4-naphthalenedione); spinochrome D (2,3,5,6,8-Pentahydroxy-1,4-naphthalenedione); and spinochrome E (hexahydroxy-1,4-naphthalenedione). The composition can include one or more additional active ingredients (e.g., vitamin, tretinoin (alltrans retinoic acid or ATRA), a glycosaminoglycan (GAG), dermal filler, Botulinum toxin, etc.) and/or one or more inactive ingredients (e.g., solvent, carrier, gelling agent, coloring agent, etc.). The composition can be in a dry, solid form (e.g., powder), or in a liquid form. Additionally, each of the above components above can independently be present in the composition in either the naturally derived form, or in a synthetically prepared form.

### PRIORITY APPLICATIONS

This application claims the benefit of priority to U.S. patent application Ser. No. 14/863,881, filed Sep. 24, 2015, which claims the benefit of priority to U.S. Provisional Patent Application Ser. No. 62/054,497, filed Sep. 24, 2014, the contents of which are hereby incorporated by reference in their entireties.

### SUMMARY

The present invention provides a composition that includes at least two (e.g., 2, 3, 4, 5, or 6) of: (a) echinochrome A (2-ethyl-3,5,6,7,8-pentahydroxy-1,4-naphthoquinone); (b) spinochrome A (2-acetyl-3,5,6,8-tetrahydroxy-1,4-naphthalenedione); (c) spinochrome B (2,3,5,7-tetrahydroxy-1,4-naphthalenedione); (d) spinochrome C (2-acetyl-3,5,6,7,8-pentahydroxy-1,4-naphthalenedione); (e) spinochrome D (2,3,5,6,8-Pentahydroxy-1,4-naphthalenedione); and (f) spinochrome E (hexahydroxy-1,4-naphthalenedione). The composition can include one or more additional active ingredients (e.g., vitamin, tretinoin (all-trans retinoic acid or ATRA), a glycosaminoglycan (GAG), dermal filler, Botulinum toxin, etc.) and/or one or more inactive ingredients (e.g., solvent, carrier, gelling agent, coloring agent, etc.). The composition can be in a dry, solid form (e.g., powder), or in a liquid form. Additionally, each of the components (a)-(f) above can independently be present in the composition in either the naturally derived form, or in a synthetically prepared form.

The sea urchin can include at least one of: Strongylocentrotus franciscanus, Strongylocentrotus purpuratus, Strongylocentrotus droebachiensis, Loxechinus albus, Evechinus Chloroticus, Strongylocentrotus droebachiensin, Strongylocentrotus intermedius, Strongylocentrotus nudus, Strongylocentrotus pallidas, Strongylocentrotus polyacanthus, Paracentrotus lividus, Lytechinus variegatus, Lytechinus variegatus, Heliocidaris erythrogramma, Centrostephanus rodgersii, and Heliocidaris tuberculate.

The present invention also provides a method that includes administering an effective amount of the composition described herein to a patient (e.g., human). The administration can be local or systemic.

The present invention also provides a method that includes topically administering an effective amount of the composition described herein to a patient (e.g., human). The topical administration of the composition is carried out to treat at least one of: a topical condition associated with aging (e.g., wrinkles and/or photoaging), acne, a topical bacterial infection, a topical fungal infection (e.g., nail fungus), and a topical viral infection (e.g., canker sores).

The present invention also provides a method that includes orally administering an effective amount of the composition described herein to a patient (e.g., human).

The present invention also provides a method of inhibiting the oxidation of another molecule (e.g., an antioxidant enzyme) present in a patient (e.g., human). The method includes administering an effective antioxidant amount of the composition described herein to the patient.

The present invention also provides a method for manufacturing a composition obtained from sea urchin. The method includes: (i) drying sea urchin to provide dried sea urchin; (ii) reducing the size of the dried sea urchin to a powder; and (iii) optionally solvent extracting the powder. The sea urchin can include at least one of: whole sea urchin, sea urchin shell, sea urchin spine, sea urchin soft tissue, and sea urchin endoskeleton. The composition can be obtained in a relatively high yield (e.g., at least about 0.1 wt. %) and/or a relatively high purity (e.g., at least about 95 wt. %). Subsequent purification of the composition can optionally be carried out, which can include, e.g., high pressure liquid chromatography (HPLC), crystallization, and/or solvent extraction.

### BRIEF DESCRIPTION OF THE DRAWINGS

Embodiments disclosed herein may be best understood by referring to the following description and accompanying drawings which illustrate such embodiments.

FIG. 1 illustrates a (block flow diagram) depicting the manufacture of a liquid extract, commercially available as T-Regalus® (FIG. 1).

FIG. 2 illustrates a (block flow diagram) depicting the manufacture of a powder form of the composition, useful, e.g., as a fertilizer (FIG. 2).

### DETAILED DESCRIPTION

Reference will now be made in detail to certain claims disclosed herein, examples of which are illustrated in the accompanying structures and formulas. While the disclosed subject matter will be described in conjunction with the enumerated claims, it will be understood that they are not intended to limit the disclosed subject matter to those claims. On the contrary, the disclosed subject matter is intended to cover all alternatives, modifications, and equivalents, which may be included within the scope of the presently disclosed subject matter as defined by the claims.

References in the specification to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The presently disclosed subject matter relates to compositions, as well as methods of making and using such compositions. When describing the compositions, as well as methods of making and using such compositions, the following terms have the following meanings, unless otherwise indicated.

As used herein, “crystallizing” includes the process of forming crystals (crystalline material), from solution.

As used herein, “size reduction” refers to reducing the size of the marine tissue (e.g., dried sea urchin shell/spine with soft tissue), typically to a powder. This can be accomplished, e.g., by shredding, milling, and/or macerating. The size reduction can be carried out, such that there is at least a 10% reduction in the average size of the solid particles, at least a 20% reduction in the average size of the solid particles, at least a 30% reduction in the average size of the solid particles, or at least a 40% reduction in the average size of the solid particles. In specific embodiments, there is up to a 70% reduction in the average size of the solid particles, up to a 50% reduction in the average size of the solid particles, or up to a 30% reduction in the average size of the solid particles.

As used herein, “drying” includes removing a substantial portion (e.g., more than 90 wt. %) of the organic solvent and water present therein. For example, the sea urchin shell/spine with soft tissue can be dried to remove a significant amount of moisture therein. This can be accomplished, e.g., by letting the marine tissue dry at ambient conditions for an extended period of time (e.g., about 48 hours), and then placed in a convection oven and heated to an elevated temperature for an extended period of time (e.g., to about 116° F. for about 72 hours). The drying can be carried out such that the final moisture content of the marine tissue is less than about 20 wt. % (e.g., about 7-12 wt. %).

As sued herein, “organic solvent” refers to a substance that is liquid at ambient conditions, wherein the compound is considered to me an organic molecule (i.e., the presence of one or more carbon atoms). The organic solvent can be polar or non-polar. Additionally, the organic solvent can be protic or non-protic. The organic solvent can include appreciable amounts of water. For example, the organic solvent can include ethanol, which itself can include water. Specifically, the organic solvent can include 95% ethanol (i.e., the organic solvent can include 95% ethanol and 5% water).

As used herein, “purifying” refers to the process of ridding a desired material of impurities. Suitable methods of purifying include, e.g., washing, solvent extraction, chromatography (e.g., HPLC), recrystallizing, and drying.

As used herein, “μg” denotes microgram, “mg” denotes milligram, “g” denotes gram, “μL” denotes microliter, “mL” denotes milliliter, “L” denotes liter, “nM” denotes nanomolar, “μM” denotes micromolar, “mM” denotes millimolar, “M” denotes molar and “nm” denotes nanometer.

### Methods of Manufacturing (Processing)

In the methods of manufacturing described herein, the steps can be carried out in any order without departing from the principles disclosed herein, except when a temporal or operational sequence is explicitly recited. Recitation in a claim to the effect that first a step is performed, and then several other steps are subsequently performed, shall be taken to mean that the first step is performed before any of the other steps, but the other steps can be performed in any suitable sequence, unless a sequence is further recited within the other steps. For example, claim elements that recite “Step A, Step B, Step C, Step D, and Step E” shall be construed to mean step A is carried out first, step E is carried out last, and steps B, C, and D can be carried out in any sequence between steps A and E, and that the sequence still falls within the literal scope of the claimed process.

Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

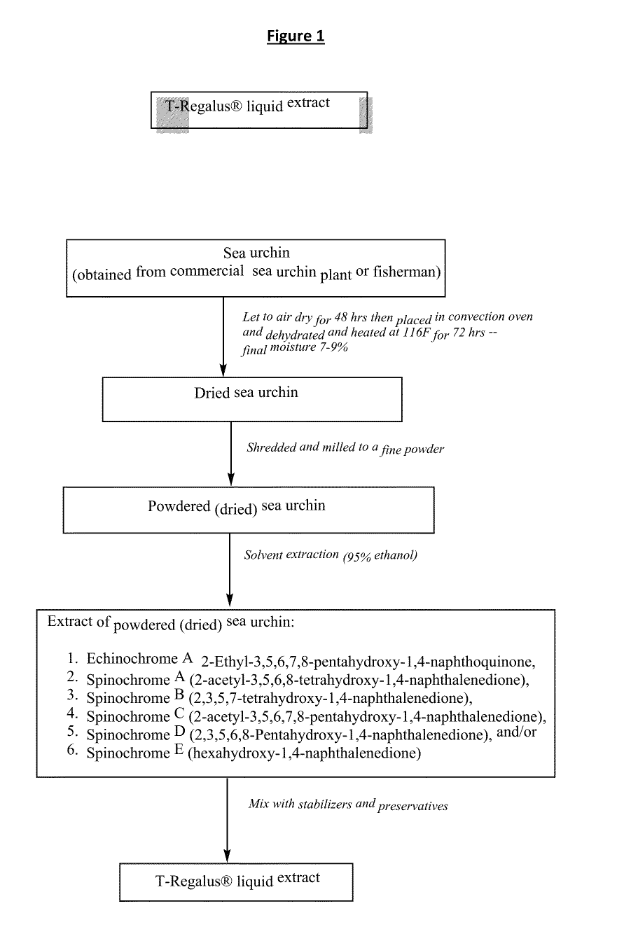

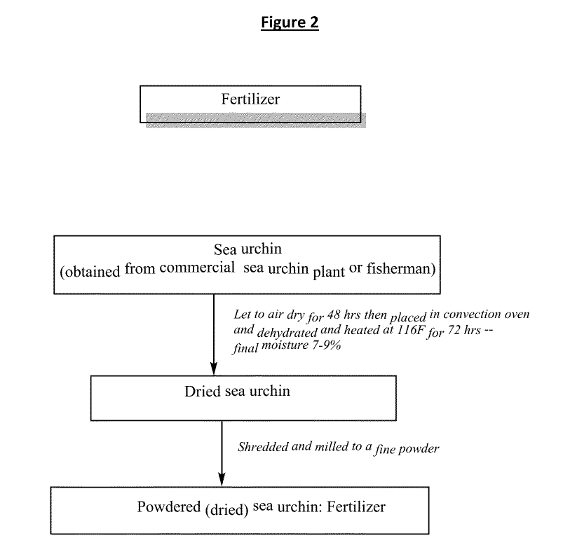

In reference to FIGS. 1 and 2, the processing methods described herein include (i) drying sea urchin to provide dried sea urchin, (ii) reducing the size of the dried sea urchin to a powder, and (iii) optionally solvent extracting the powder. The methods described herein can be carried out to obtain the compositions described herein.

The sea urchin employed in the processing methods can include the whole sea urchin, or any part thereof. For example, the sea urchin can include at least one of: whole sea urchin, sea urchin shell, sea urchin spine, sea urchin soft tissue, and sea urchin endoskeleton.

The sea urchin employed in the processing methods can include any suitable species thereof, or combination of species. For example, the sea urchin employed in the processing methods can include at least one of: Strongylocentrotus franciscanus, Strongylocentrotus purpuratus, Strongylocentrotus droebachiensis, Loxechinus albus, Evechinus Chloroticus, Strongylocentrotus droebachiensin, Strongylocentrotus intermedius, Strongylocentrotus nudus, Strongylocentrotus pallidas, Strongylocentrotus polyacanthus, Paracentrotus lividus, Lytechinus variegatus, Lytechinus variegatus, Heliocidaris erythrogramma, Centrostephanus rodgersii, and Heliocidaris tuberculate.

The drying step in the processing methods described herein can be carried out in any suitable and appropriate manner. For example, the drying step can employ a kiln, or can be carried out by sun drying.

The size reduction step in the processing methods described herein can be carried out in any suitable and appropriate manner. For example, the size reduction step can employ at least one of shredding, mulching, grinding, and milling the dried sea urchin.

The solvent extraction step in the processing methods described herein can be carried out in any suitable and appropriate manner. For example, the solvent extraction step can employ an organic solvent, such as ethanol and/or dimethyl ether. Specifically, the solvent extraction step can be carried out employing soxhlet system.

In reference to the methods shown in FIG. 1, the powdered (dried) sea urchin can be directly used in commerce. Alternatively, the powdered (dried) sea urchin can be solvent extracted (e.g., 95% ethanol and/or dimethyl ether), to obtain an extract of powdered (dried) sea urchin. In specific embodiments, the extract of powdered (dried) sea urchin can be directly used in commerce. Alternatively, the extract of powdered (dried) sea urchin can be formulated (e.g., mixed with stabilizers and/or preservatives) to provide a liquid extract (e.g., T-Regalus®).

### Pharmaceutical Formulations

The compositions disclosed herein can subsequently be formulated with conventional carriers and excipients, which will be selected in accord with ordinary practice. Tablets will contain excipients, glidants, fillers, binders and the like. Aqueous formulations are prepared in sterile form, and when intended for delivery by other than oral administration generally will be isotonic. All formulations will optionally contain excipients such as those set forth in the Handbook of Pharmaceutical Excipients, 5th Ed.; Rowe, Sheskey, and Owen, Eds.; American Pharmacists Association; Pharmaceutical Press: Washington, D C, 2006. Excipients include ascorbic acid and other antioxidants, chelating agents such as EDTA, carbohydrates such as dextrin, hydroxyalkylcellulose, hydroxyalkylmethylcellulose, stearic acid and the like. The pH of the formulations ranges from about 3 to about 11, but is ordinarily about 7 to 10.

While it is possible for the active ingredients of the composition described herein to be administered alone, it may be preferable to present them, e.g., as pharmaceutical formulations, cosmetic formulations, or plant formulations (e.g., fertilizer), etc. The formulations (for veterinary use, for human use and for plant use) disclosed herein include at least one active ingredient, as above defined, together with one or more acceptable carriers therefor and optionally other therapeutic ingredients. The carrier(s) must be “acceptable” in the sense of being compatible with the other ingredients of the formulation and physiologically innocuous to the recipient thereof.

The formulations include those suitable for the foregoing administration routes. The formulations may conveniently be presented in unit dosage form and may be prepared by any of the methods well known in the art of pharmacy. Techniques and formulations generally are found in Remington's Pharmaceutical Sciences, Mack Publishing Company, Easton, Pa., (1985). Such methods include the step of bringing into association the composition with the carrier which constitutes one or more accessory ingredients. In general the formulations are prepared by uniformly and intimately bringing into association the composition with liquid carriers or finely divided solid carriers or both, and then, if necessary, shaping the product.

Formulations disclosed herein suitable for oral administration may be presented as discrete units such as capsules, cachets or tablets each containing a predetermined amount of the composition; as a powder or granules; as a solution or a suspension in an aqueous or non-aqueous liquid; or as an oil-in-water liquid emulsion or a water-in-oil liquid emulsion. The composition may also be administered as a bolus, electuary or paste.

A tablet is made by compression or molding, optionally with one or more accessory ingredients. Compressed tablets may be prepared by compressing in a suitable machine the composition in a free-flowing form such as a powder or granules, optionally mixed with a binder, lubricant, inert diluent, preservative, surface active or dispersing agent. Molded tablets may be made by molding in a suitable machine a mixture of the powdered composition moistened with an inert liquid diluent. The tablets may optionally be coated or scored and optionally are formulated so as to provide slow or controlled release of the composition therefrom.

For administration to the eye or other external tissues e.g., mouth and skin, the formulations are preferably applied as a topical ointment or cream containing the active ingredient(s) in an amount of, for example, 0.075 to 20% w/w (including active ingredient(s) in a range between 0.1% and 20% in increments of 0.1% w/w such as 0.6% w/w, 0.7% w/w, etc.), preferably 0.2 to 15% w/w and most preferably 0.5 to 10% w/w. When formulated in an ointment, the composition may be employed with either a paraffinic or a water-miscible ointment base. Alternatively, the composition may be formulated in a cream with an oil-in-water cream base.

If desired, the aqueous phase of the cream base may include, for example, at least 30% w/w of a polyhydric alcohol, i.e. an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the composition through the skin or other affected areas. Examples of such dermal penetration enhancers include dimethyl sulphoxide and related analogs.

The oily phase of the emulsions of this disclosed subject matter may be constituted from known ingredients in a known manner. While the phase may include merely an emulsifier (otherwise known as an emulgent), it desirably includes a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations. Emulgents and emulsion stabilizers suitable for use in the formulation disclosed herein include Tween® 60, Span® 80, cetostearyl alcohol, benzyl alcohol, myristyl alcohol, glyceryl mono-stearate and sodium lauryl sulfate.

The choice of suitable oils or fats for the formulation is based on achieving the desired cosmetic properties. The cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils are used.

Pharmaceutical formulations according to the presently disclosed subject matter include one or more compositions disclosed herein together with one or more pharmaceutically acceptable carriers or excipients and optionally other therapeutic agents. Pharmaceutical formulations containing the composition may be in any form suitable for the intended method of administration. When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the composition in admixture with non-toxic pharmaceutically acceptable excipient which are suitable for manufacture of tablets are acceptable. These excipients may be, for example, inert diluents, such as calcium or sodium carbonate, lactose, lactose monohydrate, croscarmellose sodium, povidone, calcium or sodium phosphate; granulating and disintegrating agents, such as maize starch, or alginic acid; binding agents, such as cellulose, microcrystalline cellulose, starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the composition is mixed with an inert solid diluent, for example calcium phosphate or kaolin, or as soft gelatin capsules wherein the composition is mixed with water or an oil medium, such as peanut oil, liquid paraffin or olive oil.

Aqueous suspensions disclosed herein contain the active materials in admixture with excipients suitable for the manufacture of aqueous suspensions. Such excipients include a suspending agent, such as sodium carboxymethylcellulose, methylcellulose, hydroxypropyl methylcelluose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

Oil suspensions may be formulated by suspending the composition in a vegetable oil, such as arachis oil, olive oil, sesame oil or coconut oil, or in a mineral oil such as liquid paraffin. The oral suspensions may contain a thickening agent, such as beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

Dispersible powders and granules disclosed herein suitable for preparation of an aqueous suspension by the addition of water provide the composition in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions disclosed herein may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

The pharmaceutical compositions disclosed herein may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount of composition that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the composition per milliliter of solution in order that infusion of a suitable volume at a rate of about 30 mL/hr can occur.

Formulations suitable for administration to the eye include eye drops wherein the composition is dissolved or suspended in a suitable carrier, especially an aqueous solvent for the composition. The composition is preferably present in such formulations in a concentration of 0.5 to 20%, advantageously 0.5 to 10% particularly about 1.5% w/w.

Formulations suitable for topical administration in the mouth include lozenges including the composition in a flavored basis, usually sucrose and acacia or tragacanth; pastilles including the composition in an inert basis such as gelatin and glycerin, or sucrose and acacia; and mouthwashes including the composition in a suitable liquid carrier.

Formulations for rectal administration may be presented as a suppository with a suitable base including for example cocoa butter or a salicylate.

Formulations suitable for intrapulmonary or nasal administration have a particle size for example in the range of 0.1 to 500 microns (including particle sizes in a range between 0.1 and 500 microns in increments microns such as 0.5, 1, 30 microns, 35 microns, etc.), which is administered by rapid inhalation through the nasal passage or by inhalation through the mouth so as to reach the alveolar sacs. Suitable formulations include aqueous or oily solutions of the composition. Formulations suitable for aerosol or dry powder administration may be prepared according to conventional methods and may be delivered with other therapeutic agents such as compounds heretofore used in the treatment or prophylaxis of a given condition.

Formulations suitable for vaginal administration may be presented as pessaries, tampons, creams, gels, pastes, foams or spray formulations containing in addition to the composition such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations are presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the composition.

It should be understood that in addition to the ingredients particularly mentioned above the formulations of this disclosed subject matter may include other agents conventional in the art having regard to the type of formulation in question, for example those suitable for oral administration may include flavoring agents.

The disclosed subject matter further provides veterinary compositions including the composition as above described, together with a veterinary carrier therefor. Veterinary carriers are materials useful for the purpose of administering the composition and may be solid, liquid or gaseous materials which are otherwise inert or acceptable in the veterinary art and are compatible with the composition. These veterinary compositions may be administered orally, parenterally or by any other desired route.

The composition disclosed herein can also be formulated to provide controlled release of the active ingredient to allow less frequent dosing or to improve the pharmacokinetic or toxicity profile of the active ingredient. Accordingly, the disclosed subject matter also provided formulations that include the composition disclosed herein, formulated for sustained or controlled release.

Effective dose of composition depends at least on the nature of the condition being treated, toxicity, whether the composition is being used prophylactically (lower doses), the method of delivery, and the pharmaceutical formulation, and will be determined by the clinician using conventional dose escalation studies. It can be expected to be from about 0.0001 to about 100 mg/kg body weight per day. Typically, from about 0.01 to about 10 mg/kg body weight per day. More typically, from about 0.01 to about 5 mg/kg body weight per day. More typically, from about 0.05 to about 0.5 mg/kg body weight per day. For example, the daily candidate dose for an adult human of approximately 70 kg body weight will range from 1 mg to 1000 mg, preferably between 5 mg and 500 mg, and may take the form of single or multiple doses.

### Routes of Administration

The composition described herein can be administered by any route appropriate to the condition to be treated. Suitable routes include oral (including buccal and sublingual), rectal, nasal, topical, vaginal and parenteral (including subcutaneous, intramuscular, intravenous, intradermal, intrathecal and epidural), and the like. It will be appreciated that the preferred route may vary with for example the condition of the patient.

### Combination Therapy

Active ingredients (present in the compositions described herein) are also used in combination with other active ingredients. Such combinations are selected based on the condition to be treated, cross-reactivities of ingredients and pharmaco-properties of the combination.

It is also possible to combine any composition disclosed herein with one or more other active ingredients in a unitary dosage form for simultaneous or sequential administration to a patient. The combination therapy may be administered as a simultaneous or sequential regimen. When administered sequentially, the combination may be administered in two or more administrations.

The combination therapy may provide “synergy” and “synergistic effect”, i.e. the effect achieved when the active ingredients used together is greater than the sum of the effects that results from using the compounds separately. A synergistic effect may be attained when the active ingredients are: (1) co-formulated and administered or delivered simultaneously in a combined formulation; (2) delivered by alternation or in parallel as separate formulations; or (3) by some other regimen. When delivered in alternation therapy, a synergistic effect may be attained when the compounds are administered or delivered sequentially, e.g., in separate tablets, pills or capsules, or by different injections in separate syringes. In general, during alternation therapy, an effective dosage of each active ingredient is administered sequentially, i.e. serially, whereas in combination therapy, effective dosages of two or more active ingredients are administered together.

Pharmaceutical kits useful in the presently disclosed subject matter, which include a therapeutically effective amount of a pharmaceutical composition that includes a compound of component (a) and one or more compounds of component (b), in one or more sterile containers, are also within the ambit disclosed herein. Sterilization of the container may be carried out using conventional sterilization methodology well known to those skilled in the art. Component (a) and component (b) may be in the same sterile container or in separate sterile containers. The sterile containers or materials may include separate containers, or one or more multi-part containers, as desired. Component (a) and component (b), may be separate, or physically combined into a single dosage form or unit as described above. Such kits may further include, if desired, one or more of various conventional pharmaceutical kit components, such as for example, one or more pharmaceutically acceptable carriers, additional vials for mixing the components, etc., as will be readily apparent to those skilled in the art. Instructions, either as inserts or as labels, indicating quantities of the components to be administered, guidelines for administration, and/or guidelines for mixing the components, may also be included in the kit.

Specific ranges, values, and embodiments provided below are for illustration purposes only and do not otherwise limit the scope of the invention, as defined by the claims. The specific ranges, values, and embodiments described below encompass all combinations and sub-combinations of each disclosed range, value, and embodiment, whether or not expressly described as such.

### Specific Ranges, Values, and Embodiments

In specific embodiments, the echinochrome A is present in up to about 50 wt. % of the composition.

In specific embodiments, the echinochrome A is present in up to about 40 wt. % of the composition.

In specific embodiments, the echinochrome A is present in up to about 30 wt. % of the composition.

In specific embodiments, the echinochrome A is present in at least about 15 wt. % of the composition.

In specific embodiments, the echinochrome A is present in at least about 20 wt. % of the composition.

In specific embodiments, the echinochrome A is present in about 15 wt. % to about 35 wt. % of the composition.

In specific embodiments, the echinochrome A is present in about 20 wt. % to about 30 wt. % of the composition.

In specific embodiments, the spinochrome A is present in up to about 50 wt. % of the composition.

In specific embodiments, the spinochrome A is present in up to about 40 wt. % of the composition.

In specific embodiments, the spinochrome A is present in up to about 30 wt. % of the composition.

In specific embodiments, the spinochrome A is present in at least about 15 wt. % of the composition.

In specific embodiments, the spinochrome A is present in at least about 20 wt. % of the composition.

In specific embodiments, the spinochrome A is present in about 15 wt. % to about 35 wt. % of the composition.

In specific embodiments, the spinochrome A is present in about 20 wt. % to about 30 wt. % of the composition.

In specific embodiments, the spinochrome B is present in up to about 50 wt. % of the composition.

In specific embodiments, the spinochrome B is present in up to about 40 wt. % of the composition.

In specific embodiments, the spinochrome B is present in up to about 30 wt. % of the composition.

In specific embodiments, the spinochrome B is present in at least about 15 wt. % of the composition.

In specific embodiments, the spinochrome B is present in at least about 20 wt. % of the composition.

In specific embodiments, the spinochrome B is present in about 15 wt. % to about 35 wt. % of the composition.

In specific embodiments, the spinochrome B is present in about 20 wt. % to about 30 wt. % of the composition.

In specific embodiments, the spinochrome C is present in up to about 50 wt. % of the composition.

In specific embodiments, the spinochrome C is present in up to about 40 wt. % of the composition.

In specific embodiments, the spinochrome C is present in up to about 30 wt. % of the composition.

In specific embodiments, the spinochrome C is present in at least about 15 wt. % of the composition.

In specific embodiments, the spinochrome C is present in at least about 20 wt. % of the composition.

In specific embodiments, the spinochrome C is present in about 15 wt. % to about 35 wt. % of the composition.

In specific embodiments, the spinochrome C is present in about 20 wt. % to about 30 wt. % of the composition.

In specific embodiments, the spinochrome D is present in up to about 50 wt. % of the composition.

In specific embodiments, the spinochrome D is present in up to about 40 wt. % of the composition.

In specific embodiments, the spinochrome D is present in up to about 30 wt. % of the composition.

In specific embodiments, the spinochrome D is present in at least about 15 wt. % of the composition.

In specific embodiments, the spinochrome D is present in at least about 20 wt. % of the composition.

In specific embodiments, the spinochrome D is present in about 15 wt. % to about 35 wt. % of the composition.

In specific embodiments, the spinochrome D is present in about 20 wt. % to about 30 wt. % of the composition.

In specific embodiments, the spinochrome E is present in up to about 50 wt. % of the composition.

In specific embodiments, the spinochrome E is present in up to about 40 wt. % of the composition.

In specific embodiments, the spinochrome E is present in up to about 30 wt. % of the composition.

In specific embodiments, the spinochrome E is present in at least about 15 wt. % of the composition.

In specific embodiments, the spinochrome E is present in at least about 20 wt. % of the composition.

In specific embodiments, the spinochrome E is present in about 15 wt. % to about 35 wt. % of the composition.

In specific embodiments, the spinochrome E is present in about 20 wt. % to about 30 wt. % of the composition.

In specific embodiments, the composition includes two of echinochrome A, spinochrome A, spinochrome B, spinochrome C, spinochrome D, and spinochrome E.

In specific embodiments, the composition includes three of echinochrome A, spinochrome A, spinochrome B, spinochrome C, spinochrome D, and spinochrome E.

In specific embodiments, the composition includes four of echinochrome A, spinochrome A, spinochrome B, spinochrome C, spinochrome D, and spinochrome E.

In specific embodiments, the composition includes five of echinochrome A, spinochrome A, spinochrome B, spinochrome C, spinochrome D, and spinochrome E.

In specific embodiments, the composition includes six of echinochrome A, spinochrome A, spinochrome B, spinochrome C, spinochrome D, and spinochrome E.

In specific embodiments, the composition (with respect to the aggregate of echinochrome A, spinochrome A, spinochrome B, spinochrome C, spinochrome D, and/or spinochrome E, present therein) has a purity of at least about 75 wt. %.

In specific embodiments, the composition (with respect to the aggregate of echinochrome A, spinochrome A, spinochrome B, spinochrome C, spinochrome D, and/or spinochrome E, present therein) has a purity of at least about 80 wt. %.

In specific embodiments, the composition (with respect to the aggregate of echinochrome A, spinochrome A, spinochrome B, spinochrome C, spinochrome D, and/or spinochrome E, present therein) has a purity of at least about 85 wt. %.

In specific embodiments, the composition (with respect to the aggregate of echinochrome A, spinochrome A, spinochrome B, spinochrome C, spinochrome D, and/or spinochrome E, present therein) has a purity of at least about 90 wt. %.

In specific embodiments, the composition (with respect to the aggregate of echinochrome A, spinochrome A, spinochrome B, spinochrome C, spinochrome D, and/or spinochrome E, present therein) has a purity of at least about 95 wt. %.

In specific embodiments, the composition (with respect to the aggregate of echinochrome A, spinochrome A, spinochrome B, spinochrome C, spinochrome D, and/or spinochrome E, present therein) has a purity of at least about 98 wt. %.

In specific embodiments, the composition (with respect to the aggregate of echinochrome A, spinochrome A, spinochrome B, spinochrome C, spinochrome D, and/or spinochrome E, present therein) has a purity of at least about 99 wt. %.

In specific embodiments, the composition is in the form of a solid, having a particle size of about D₅₀ 10-1,000 μm.

In specific embodiments, the composition is in the form of a solid, having a particle size of about D₅₀ 50-1,000 μm.

In specific embodiments, the composition is in the form of a solid, having a particle size of about D₅₀ 10-500 μm.

In specific embodiments, the composition is in the form of a solid, having a particle size of about D₅₀ 100-1,000 μm.

In specific embodiments, the composition is in the form of a solid, having a particle size of about D₅₀ 10-250 μm.

In specific embodiments, the composition is in the form of a solid, having a particle size of less than about D₅₀ 1,000 μm.

In specific embodiments, the composition is in the form of a solid, having a particle size of less than about D₅₀ 750 μm.

In specific embodiments, the composition is in the form of a solid, having a particle size of less than about D₅₀ 500 μm.

In specific embodiments, the composition is in the form of a solid, having a particle size of about D₉₀ 10-1,000 μm.

In specific embodiments, the composition is in the form of a solid, having a particle size of about D₉₀ 50-1,000 μm.

In specific embodiments, the composition is in the form of a solid, having a particle size of about D₉₀ 10-500 μm.

In specific embodiments, the composition is in the form of a solid, having a particle size of about D₉₀ 100-1,000 μm.

In specific embodiments, the composition is in the form of a solid, having a particle size of about D₉₀ 10-250 μm.

In specific embodiments, the composition is in the form of a solid, having a particle size of less than about D₉₀ 1,000 μm.

In specific embodiments, the composition is in the form of a solid, having a particle size of less than about D₉₀ 750 μm.

In specific embodiments, the composition is in the form of a solid, having a particle size of less than about D₉₀ 500 μm.

In specific embodiments, the composition is in the form of a solid, having a final moisture content of less that about 25 wt. %.

In specific embodiments, the composition is in the form of a solid, having a final moisture content of less that about 20 wt. %.

In specific embodiments, the composition is in the form of a solid, having a final moisture content of less that about 15 wt. %.

In specific embodiments, the composition is in the form of a solid, having a final moisture content of less that about 10 wt. %.

In specific embodiments, the composition is in the form of a solid, having a final moisture content of less that about 5 wt. %.

In specific embodiments, the composition is in the form of a solid, having a final moisture content of less that about 2.5 wt. %.

In specific embodiments, the composition is in the form of a solid, having a final moisture content of less that about 1 wt. %.

In specific embodiments, the composition is in the form of a solid, having a final moisture content of less that about 0.5 wt. %.

In specific embodiments, the composition is essentially free of marine matter (e.g., sea urchin tissue or cellular material).

In specific embodiments, the composition includes less than about 10 wt. % marine matter (e.g., sea urchin tissue or cellular material).

In specific embodiments, the composition includes less than about 5 wt. % marine matter (e.g., sea urchin tissue or cellular material).

In specific embodiments, the composition includes less than about 1 wt. % marine matter (e.g., sea urchin tissue or cellular material).

In specific embodiments, the composition includes less than about 0.5 wt. % marine matter (e.g., sea urchin tissue or cellular material).

In specific embodiments, the composition includes less than about 0.1 wt. % marine matter (e.g., sea urchin tissue or cellular material).

In specific embodiments, the solvent extraction step in the processing method employs an organic solvent. In further specific embodiments, the organic solvent is cold (e.g., less than about 25° C.). In further specific embodiments, the organic solvent is hot (e.g., greater than about 25° C.).

In specific embodiments, the solvent extraction step in the processing method employs an organic solvent such as ethanol. In specific embodiments, the solvent extraction step in the processing method employs an organic solvent such as dimethyl ether.

In specific embodiments, the solvent extraction step in the processing method includes a single solvent. In specific embodiments, the solvent extraction step in the processing method includes multiple solvents.

In specific embodiments, the solvent extraction step in the processing method is carried out multiple times, each with a different solvent, or combination of solvents.

In specific embodiments, the solvent extraction step in the processing method is carried out employing a soxhlet system.

In specific embodiments, the processing method yields up to about 1 gram of composition (with respect to the aggregate of echinochrome A, spinochrome A, spinochrome B, spinochrome C, spinochrome D, and/or spinochrome E), based upon 1 kilogram of sea urchin employed.

In specific embodiments, the processing method yields up to about 1.25 grams of composition (with respect to the aggregate of echinochrome A, spinochrome A, spinochrome B, spinochrome C, spinochrome D, and/or spinochrome E), based upon 1 kilogram of sea urchin employed.

In specific embodiments, the processing method yields at least about 1 gram of composition (with respect to the aggregate of echinochrome A, spinochrome A, spinochrome B, spinochrome C, spinochrome D, and/or spinochrome E), based upon 1 kilogram of sea urchin employed.

In specific embodiments, the processing method yields at least about 1.25 grams of composition (with respect to the aggregate of echinochrome A, spinochrome A, spinochrome B, spinochrome C, spinochrome D, and/or spinochrome E), based upon 1 kilogram of sea urchin employed.

In specific embodiments, the processing method yields at least about 2 grams of composition (with respect to the aggregate of echinochrome A, spinochrome A, spinochrome B, spinochrome C, spinochrome D, and/or spinochrome E), based upon 1 kilogram of sea urchin employed.

### Enumerated Embodiments

Specific enumerated embodiments [1] to [77] provided below are for illustration purposes only, and do not otherwise limit the scope of the disclosed subject matter, as defined by the claims. These enumerated embodiments encompass all combinations, sub-combinations, and multiply referenced (e.g., multiply dependent) combinations described therein.

[1.] The present invention provides a composition that includes at least two of:

(a) echinochrome A (2-ethyl-3,5,6,7,8-pentahydroxy-1,4-naphthoquinone);

(b) spinochrome A (2-acetyl-3,5,6,8-tetrahydroxy-1,4-naphthalenedione);

(c) spinochrome B (2,3,5,7-tetrahydroxy-1,4-naphthalenedione);

(d) spinochrome C (2-acetyl-3,5,6,7,8-pentahydroxy-1,4-naphthalenedione);

(e) spinochrome D (2,3,5,6,8-Pentahydroxy-1,4-naphthalenedione); and

(f) spinochrome E (hexahydroxy-1,4-naphthalenedione).

[2.] The present invention also provides the composition as described in embodiment [1], that includes at least three of (a)-(f).
[3.] The present invention also provides the composition as described in embodiment [1], that includes at least four of (a)-(f).
[4.] The present invention also provides the composition as described in embodiment [1], that includes at least five of (a)-(f).
[5.] The present invention also provides the composition as described in embodiment [1], that includes each of (a)-(f).
[6.] The present invention also provides the composition as described in any of the embodiments above, wherein the echinochrome A is present in up to about 50 wt. % of the composition.
[7.] The present invention also provides the composition as described in any of the embodiments above, wherein the echinochrome A is present in about 15 wt. % to about 35 wt. % of the composition.
[8.] The present invention also provides the composition as described in any of the embodiments above, wherein the spinochrome A is present in up to about 50 wt. % of the composition.
[9.] The present invention also provides the composition as described in any of the embodiments above, wherein the spinochrome A is present in about 15 wt. % to about 35 wt. % of the composition.
[10.] The present invention also provides the composition as described in any of the embodiments above, wherein the spinochrome B is present in up to about 50 wt. % of the composition.
[11.] The present invention also provides the composition as described in any of the embodiments above, wherein the spinochrome B is present in about 15 wt. % to about 35 wt. % of the composition.
[12.] The present invention also provides the composition as described in any of the embodiments above, wherein the spinochrome C is present in up to about 50 wt. % of the composition.
[13.] The present invention also provides the composition as described in any of the embodiments above, wherein the spinochrome C is present in about 15 wt. % to about 35 wt. % of the composition.
[14.] The present invention also provides the composition as described in any of the embodiments above, wherein the spinochrome D is present in up to about 50 wt. % of the composition.
[15.] The present invention also provides the composition as described in any of the embodiments above, wherein the spinochrome D is present in about 15 wt. % to about 35 wt. % of the composition.
[16.] The present invention also provides the composition as described in any of the embodiments above, wherein the spinochrome E is present in up to about 50 wt. % of the composition.
[17.] The present invention also provides the composition as described in any of the embodiments above, wherein the spinochrome E is present in about 15 wt. % to about 35 wt. % of the composition.
[18.] The present invention also provides the composition as described in any of the embodiments above, having a purity of at least about 95 wt. %.
[19.] The present invention also provides the composition as described in any of the embodiments above, having a purity of at least about 98 wt. %.
[20.] The present invention also provides the composition as described in any of the embodiments above, further including one or more additional active ingredients.
[21.] The present invention also provides the composition as described in any of the embodiments above, which is in a dry, solid form.
[22.] The present invention also provides the composition as described in any of the embodiments above, which is in a solid form having a particle size of about D₅₀ 10-1,000 μm.
[23.] The present invention also provides the composition as described in any of the embodiments above, which is in a powder form.
[24.] The present invention also provides the composition as described in any of the embodiments above, which is in a solid form, and is formulated as a fertilizer.
[25.] The present invention also provides the composition as described in any of the embodiments above, which is in a solid form having a final moisture content of less that about 15 wt. %.
[26.] The present invention also provides the composition as described in any of the embodiments above, which is in a liquid form.
[27.] The present invention also provides the composition as described in any of the embodiments above, which is essentially free of marine plant matter.
[28.] The present invention also provides the composition as described in any of the embodiments above, further including a carrier.
[29.] The present invention also provides the composition as described in any of the embodiments above, further including a pharmaceutically acceptable carrier.
[30.] The present invention also provides the composition as described in any of the embodiments above, further including a cosmetically acceptable carrier.
[31.] The present invention also provides the composition as described in any of the embodiments above, further including at least one of an:

(a) adsorbent,

(b) suspending agent,

(c) diluent,

(d) disintegrant,

(e) glidant,

(f) binder,

(g) acidifying agent,

(h) basifying agent,

(i) antioxidant or free radical scavenger,

(j) buffering agent,

(k) chelating agent,

(l) fragrance,

(m) waxy substance,

(n) oil,

(o) lubricant,

(p) humectant,

(q) emollient,

(r) coloring agent,

(s) dye,

(t) penetration enhancer,

(u) gelling agent

(v) stabilizer,

(w) preservative,

(x) emulsifier,

(y) solvent, and

(z) co-solvent.

[32.] The present invention also provides the composition as described in any of the embodiments above, further including at least one of a Vitamin, tretinoin (all-trans retinoic acid or ATRA), a glycosaminoglycan (GAG), dermal filler, and Botulinum toxin.
[33.] The present invention also provides the composition as described in any of the embodiments above, formulated for oral administration.
[34.] The present invention also provides the composition as described in any of the embodiments above, formulated for topical administration.
[35.] The present invention also provides the composition as described in any of the embodiments above, formulated for injectable administration.
[36.] The present invention also provides the composition as described in any of the embodiments above, which is a cream, lotion, ointment, gel, topical adhesive patch, or foam, suitable for topical administration.
[37.] The present invention also provides the composition as described in any of the embodiments above, which is a cosmetic composition.
[38.] The present invention also provides the composition as described in any of the embodiments above, which is a pharmaceutical composition.
[39.] The present invention also provides the composition as described in any of the embodiments above, which is a liquid.
[40.] The present invention also provides the composition as described in any of the embodiments above, which is a liquid, suitable for human consumption.
[41.] The present invention also provides the composition as described in any of the embodiments above, wherein at least one of the:

(a) echinochrome A (2-ethyl-3,5,6,7,8-pentahydroxy-1,4-naphthoquinone);

(b) spinochrome A (2-acetyl-3,5,6,8-tetrahydroxy-1,4-naphthalenedione);

(c) spinochrome B (2,3,5,7-tetrahydroxy-1,4-naphthalenedione);

(d) spinochrome C (2-acetyl-3,5,6,7,8-pentahydroxy-1,4-naphthalenedione);

(e) spinochrome D (2,3,5,6,8-Pentahydroxy-1,4-naphthalenedione); and

(f) spinochrome E (hexahydroxy-1,4-naphthalenedione)

is naturally derived.
[42.] The present invention also provides the composition as described in any of the embodiments above, wherein each of the:

(a) echinochrome A (2-ethyl-3,5,6,7,8-pentahydroxy-1,4-naphthoquinone);

(b) spinochrome A (2-acetyl-3,5,6,8-tetrahydroxy-1,4-naphthalenedione);

(c) spinochrome B (2,3,5,7-tetrahydroxy-1,4-naphthalenedione);

(d) spinochrome C (2-acetyl-3,5,6,7,8-pentahydroxy-1,4-naphthalenedione);

(e) spinochrome D (2,3,5,6,8-Pentahydroxy-1,4-naphthalenedione); and

(f) spinochrome E (hexahydroxy-1,4-naphthalenedione)

is naturally derived.
[43.] The present invention also provides the composition as described in any of the embodiments above, wherein at least one of the:

(a) echinochrome A (2-ethyl-3,5,6,7,8-pentahydroxy-1,4-naphthoquinone);

(b) spinochrome A (2-acetyl-3,5,6,8-tetrahydroxy-1,4-naphthalenedione);

(c) spinochrome B (2,3,5,7-tetrahydroxy-1,4-naphthalenedione);

(d) spinochrome C (2-acetyl-3,5,6,7,8-pentahydroxy-1,4-naphthalenedione);

(e) spinochrome D (2,3,5,6,8-Pentahydroxy-1,4-naphthalenedione); and

(f) spinochrome E (hexahydroxy-1,4-naphthalenedione)

is synthetically prepared.
[44.] The present invention also provides the composition as described in any of the embodiments above, wherein each of the:

(a) echinochrome A (2-ethyl-3,5,6,7,8-pentahydroxy-1,4-naphthoquinone);

(b) spinochrome A (2-acetyl-3,5,6,8-tetrahydroxy-1,4-naphthalenedione);

(c) spinochrome B (2,3,5,7-tetrahydroxy-1,4-naphthalenedione);

(d) spinochrome C (2-acetyl-3,5,6,7,8-pentahydroxy-1,4-naphthalenedione);

(e) spinochrome D (2,3,5,6,8-Pentahydroxy-1,4-naphthalenedione); and

(f) spinochrome E (hexahydroxy-1,4-naphthalenedione)

is synthetically prepared.
[45.] The present invention also provides a method that includes orally administering an effective amount of the composition of any one of the above embodiments to a patient.
[46.] The present invention also provides the method that includes topically administering an effective amount of the composition of any one of the above embodiments to a patient.
[47.] The present invention also provides the method as described in embodiment [46], which includes a method of treating at least one of:

a topical condition associated with aging;

acne;

a topical bacterial infection;

a topical fungal infection; and

a topical viral infection.

[48.] The present invention also provides the method as described in embodiment [46], wherein the topical condition associated with aging includes at least one of wrinkles and photoaging.
[49.] The present invention also provides the method as described in embodiment [47], wherein the topical fungal infection includes a nail fungus.
[50.] The present invention also provides the method as described in embodiment [47], wherein the topical viral infection includes canker sores.
[51.] The present invention also provides a method of inhibiting the oxidation of another molecule present in a patient, the method including administering an effective antioxidant amount of the composition of any one of the above claims to a patient.
[52.] The present invention also provides the method as described in embodiment [51], wherein the inhibiting the oxidation of another molecule present in the patient includes inhibiting an antioxidant enzyme.
[53.] The present invention also provides the method as described in any one of embodiments [51]-[52], wherein the route of administration is topical.
[54.] The present invention also provides the method as described in any one of embodiments [51]-[52], wherein the route of administration is oral.
[55.] The present invention also provides the method as described in any one of the above embodiments, wherein the patient is a human.
[56.] The present invention also provides a method that includes:

(i) drying sea urchin to provide dried sea urchin;

(ii) reducing the size of the dried sea urchin to a powder; and

(iii) optionally solvent extracting the powder.

[57.] The present invention also provides the method as described in embodiment [56], which is a method of obtaining at least one of:

(a) echinochrome A (2-ethyl-3,5,6,7,8-pentahydroxy-1,4-naphthoquinone);

(b) spinochrome A (2-acetyl-3,5,6,8-tetrahydroxy-1,4-naphthalenedione);

(c) spinochrome B (2,3,5,7-tetrahydroxy-1,4-naphthalenedione);

(d) spinochrome C (2-acetyl-3,5,6,7,8-pentahydroxy-1,4-naphthalenedione);

(e) spinochrome D (2,3,5,6,8-Pentahydroxy-1,4-naphthalenedione); and

(f) spinochrome E (hexahydroxy-1,4-naphthalenedione).

[58.] The present invention also provides the method as described in embodiment [56], which is a method of obtaining at least two of:

(a) echinochrome A (2-ethyl-3,5,6,7,8-pentahydroxy-1,4-naphthoquinone);

(b) spinochrome A (2-acetyl-3,5,6,8-tetrahydroxy-1,4-naphthalenedione);

(c) spinochrome B (2,3,5,7-tetrahydroxy-1,4-naphthalenedione);

(d) spinochrome C (2-acetyl-3,5,6,7,8-pentahydroxy-1,4-naphthalenedione);

(e) spinochrome D (2,3,5,6,8-Pentahydroxy-1,4-naphthalenedione); and

(f) spinochrome E (hexahydroxy-1,4-naphthalenedione).

[59.] The present invention also provides the method as described in embodiment [56], which is a method of obtaining at least three of:

(a) echinochrome A (2-ethyl-3,5,6,7,8-pentahydroxy-1,4-naphthoquinone);

(b) spinochrome A (2-acetyl-3,5,6,8-tetrahydroxy-1,4-naphthalenedione);

(c) spinochrome B (2,3,5,7-tetrahydroxy-1,4-naphthalenedione);

(d) spinochrome C (2-acetyl-3,5,6,7,8-pentahydroxy-1,4-naphthalenedione);

(e) spinochrome D (2,3,5,6,8-Pentahydroxy-1,4-naphthalenedione); and

(f) spinochrome E (hexahydroxy-1,4-naphthalenedione).

[60.] The present invention also provides the method as described in embodiment [56], which is a method of obtaining at least four of:

(a) echinochrome A (2-ethyl-3,5,6,7,8-pentahydroxy-1,4-naphthoquinone);

(b) spinochrome A (2-acetyl-3,5,6,8-tetrahydroxy-1,4-naphthalenedione);

(c) spinochrome B (2,3,5,7-tetrahydroxy-1,4-naphthalenedione);

(d) spinochrome C (2-acetyl-3,5,6,7,8-pentahydroxy-1,4-naphthalenedione);

(e) spinochrome D (2,3,5,6,8-Pentahydroxy-1,4-naphthalenedione); and

(f) spinochrome E (hexahydroxy-1,4-naphthalenedione).

[61.] The present invention also provides the method as described in embodiment [56], which is a method of obtaining at least five of:

(a) echinochrome A (2-ethyl-3,5,6,7,8-pentahydroxy-1,4-naphthoquinone);

(b) spinochrome A (2-acetyl-3,5,6,8-tetrahydroxy-1,4-naphthalenedione);

(c) spinochrome B (2,3,5,7-tetrahydroxy-1,4-naphthalenedione);

(d) spinochrome C (2-acetyl-3,5,6,7,8-pentahydroxy-1,4-naphthalenedione);

(e) spinochrome D (2,3,5,6,8-Pentahydroxy-1,4-naphthalenedione); and

(f) spinochrome E (hexahydroxy-1,4-naphthalenedione).

[62.] The present invention also provides the method as described in embodiment [56], which is a method of obtaining each of:

(a) echinochrome A (2-ethyl-3,5,6,7,8-pentahydroxy-1,4-naphthoquinone);

(b) spinochrome A (2-acetyl-3,5,6,8-tetrahydroxy-1,4-naphthalenedione);

(c) spinochrome B (2,3,5,7-tetrahydroxy-1,4-naphthalenedione);

(d) spinochrome C (2-acetyl-3,5,6,7,8-pentahydroxy-1,4-naphthalenedione);

(e) spinochrome D (2,3,5,6,8-Pentahydroxy-1,4-naphthalenedione); and

(f) spinochrome E (hexahydroxy-1,4-naphthalenedione).

[63.] The present invention also provides the method as described in any one of embodiments [56]-[62], wherein the sea urchin includes at least one of:

whole sea urchin,

sea urchin shell,

sea urchin spine,

sea urchin soft tissue, and

sea urchin endoskeleton.

[64.] The present invention also provides the method as described in any one of embodiments [56]-[63], wherein the sea urchin includes at least one of:

Strongylocentrotus franciscanus;

Strongylocentrotus purpuratus;

Strongylocentrotus droebachiensis;

Loxechinus albus;

Evechinus Chloroticus;

Strongylocentrotus droebachiensin;

Strongylocentrotus intermedius;

Strongylocentrotus nudus;

Strongylocentrotus pollidas;

Strongylocentrotus polyacanthus;

Paracentrotus lividus;

Lytechinus variegatus;

Lytechinus variegotus;

Heliocidaris erythrogramma;

Centrostephanus rodgersii; and

Heliocidaris tuberculate.

[65.] The present invention also provides the method as described in any one of embodiments [56]-[64], wherein the drying of the sea urchin is carried out employing a kiln.
[66.] The present invention also provides the method as described in any one of embodiments [56]-[64], wherein the drying of the sea urchin is carried out by sun drying.
[67.] The present invention also provides the method as described in any one of embodiments [56]-[66], wherein the drying of the sea urchin is carried out to a final moisture content of less that about 15 wt. %.
[68.] The present invention also provides the method as described in any one of embodiments [56]-[66], wherein the drying of the sea urchin is carried out to a final moisture content of about 7-12 wt. %.
[69.] The present invention also provides the method as described in any one of embodiments [56]-[68], wherein the reducing the size of the dried sea urchin, to a powder, includes at least one of shredding, mulching, grinding, and milling.
[70.] The present invention also provides the method as described in any one of embodiments [56]-[69], wherein the solvent extraction employs an organic solvent.
[71.] The present invention also provides the method as described in any one of embodiments [56]-[70], wherein the solvent extraction employs ethanol.
[72.] The present invention also provides the method as described in any one of embodiments [56]-[71], wherein the yield is at least about 1 gram of components (a)-(f) in the aggregate, based upon 1 kilogram of sea urchin employed.
[73.] The present invention also provides the method as described in any one of embodiments [56]-[71], wherein the yield is up to about 1.25 grams of components (a)-(f) in the aggregate, based upon 1 kilogram of sea urchin employed.
[74.] The present invention also provides the method as described in any one of embodiments [56]-[71], wherein the yield is about 1-1.21 grams of components (a)-(f) in the aggregate, based upon 1 kilogram of sea urchin employed.
[75.] The present invention also provides the method as described in any one of embodiments [56]-[74], further including after step (iii), the step of (iv) purifying the aggregate of components (a)-(f).
[76.] The present invention also provides the method as described in any one of embodiments [56]-[74], further including after step (iii), the step of (iv) isolating any one or more of components (a)-(f).
[77.] The present invention also provides the method as described in any one of embodiments [56]-[76], wherein the purifying or isolating includes at least one of high pressure liquid chromatography (HPLC), crystallization, and solvent extraction.

All publications, patents, and patent applications are incorporated herein by reference. While in the foregoing specification this disclosed subject matter has been described in relation to certain preferred embodiments thereof, and many details have been set forth for purposes of illustration, it will be apparent to those skilled in the art that the disclosed subject matter is susceptible to additional embodiments and that certain of the details described herein may be varied considerably without departing from the basic principles disclosed herein.

What is claimed is:

1. A composition comprising:
(a) echinochrome A (2-ethyl-3,5,6,7,8-pentahydroxy-1,4-naphthoquinone);
(b) spinochrome A (2-acetyl-3,5,6,8-tetrahydroxy-1,4-naphthalenedione);
(c) spinochrome B (2,3,5,7-tetrahydroxy-1,4-naphthalenedione);
(d) spinochrome C (2-acetyl-3,5,6,7,8-pentahydroxy-1,4-naphthalenedione);
(e) spinochrome D (2,3,5,6,8-Pentahydroxy-1,4-naphthalenedione); and
(f) spinochrome E (hexahydroxy-1,4-naphthalenedione).

2. The composition of claim 1, having a purity of at least about 98 wt. %.

3. The composition of claim 1, which is in a solid form having a particle size of about D₅₀ 10-1,000 μm.

4. The composition of claim 1, which is in a powder form.

5. The composition of claim 1, which is in a solid form, and is formulated as a fertilizer.

6. The composition of claim 1, which is in a liquid form.

7. The composition of claim 1, which is essentially free of marine tissue.

8. The composition of claim 1, further comprising a carrier.

9. The composition of claim 1, further comprising at least one of an:
(a) adsorbent,
(b) suspending agent,
(c) diluent,
(d) disintegrant,
(e) glidant,
(f) binder,
(g) acidifying agent,
(h) basifying agent,
(i) antioxidant or free radical scavenger,
(j) buffering agent,
(k) chelating agent,
(l) fragrance,
(m) waxy substance,
(n) oil,
(o) lubricant,
(p) humectant,
(q) emollient,
(r) coloring agent,
(s) dye,
(t) penetration enhancer,
(u) gelling agent
(v) stabilizer,
(w) preservative,
(x) emulsifier,
(y) solvent, and
(z) co-solvent.

10. The composition of claim 1, further comprising at least one of a Vitamin, tretinoin (all-trans retinoic acid or ATRA), a glycosaminoglycan (GAG), dermal filler, and Botulinum toxin.

11. A method comprising orally administering an effective amount of the composition of claim 1 to a patient.

12. A method comprising topically administering an effective amount of the composition of claim 1 to a patient.

13. The method of claim 12, which comprises a method of treating at least one of:
a topical condition associated with aging;
acne;
a topical bacterial infection;
a topical fungal infection; and
a topical viral infection.

14. The method of claim 13, wherein the topical condition associated with aging comprises at least one of wrinkles and photoaging.

15. The method of claim 13, wherein the topical fungal infection comprises a nail fungus.

16. The method of claim 13, wherein the topical viral infection comprises canker sores.

17. A method of inhibiting the oxidation of another molecule present in a patient, the method comprising administering an effective antioxidant amount of the composition of claim 1 to a patient.

18. The method of claim 17, wherein the inhibiting the oxidation of another molecule present in the patient comprises inhibiting an antioxidant enzyme.

19. A method comprising:
(i) drying sea urchin to provide dried sea urchin;
(ii) reducing the size of the dried sea urchin to a powder; and
(iii) optionally solvent extracting the powder.

20. The method of claim 19, which is a method of obtaining:
(a) echinochrome A (2-ethyl-3,5,6,7,8-pentahydroxy-1,4-naphthoquinone);
(b) spinochrome A (2-acetyl-3,5,6,8-tetrahydroxy-1,4-naphthalenedione);
(c) spinochrome B (2,3,5,7-tetrahydroxy-1,4-naphthalenedione);
(d) spinochrome C (2-acetyl-3,5,6,7,8-pentahydroxy-1,4-naphthalenedione);
(e) spinochrome D (2,3,5,6,8-Pentahydroxy-1,4-naphthalenedione); and
(f) spinochrome E (hexahydroxy-1,4-naphthalenedione).

21. The method of claim 19, wherein the sea urchin comprises at least one of:
whole sea urchin,
sea urchin shell,
sea urchin spine,
sea urchin soft tissue, and
sea urchin endoskeleton.

22. The method of claim 19, wherein the sea urchin comprises at least one of:
Strongylocentrotus francisconus;
Strongylocentrotus purpuratus;
Strongylocentrotus droebachiensis;
Loxechinus albus;
Evechinus Chloroticus;
Strongylocentrotus droebachiensin;
Strongylocentrotus intermedius;
Strongylocentrotus nudus;
Strongylocentrotus pallidas;
Strongylocentrotus polyacanthus;
Paracentrotus lividus;
Lytechinus variegatus;
Lytechinus variegatus;
Heliocidaris erythrogramma;
Centrostephanus rodgersii; and
Heliocidaris tuberculate.

23. The method of claim 19, wherein the drying of the sea urchin is carried out employing a kiln.

24. The method of claim 19, wherein the drying of the sea urchin is carried out by sun drying.

25. The method of claim 19, wherein the reducing the size of the dried sea urchin, to a powder, comprises at least one of shredding, mulching, grinding, and milling.

26. The method of claim 19, wherein the solvent extraction employs an organic solvent.

27. The method of claim 19, wherein the solvent extraction employs ethanol.

28. The method of claim 19, wherein the yield is at least about 1 gram of components (a)-(f) in the aggregate, based upon 1 kilogram of sea urchin employed.

29. The method of claim 19, further comprising after step (iii), the step of (iv) purifying the aggregate of components (a)-(f).

30. The method of claim 19, further comprising after step (iii), the step of (iv) isolating any one or more of components (a)-(f).
